(19) United States
(12) Patent Application Publication
(10) Pub. No.: US 2024/0415688 A1
(43) Pub. Date: Dec. 19, 2024

(54) Brace Having Interchangeable Therapeutic Modalities

(71) Applicant: YUR Brace, LLC, Dakota Dunes, SD (US)

(72) Inventors: Steven Jay Meyer, Dakota Dunes, SD (US); Judson A. Herrig, Elko New Market, MN (US)

(73) Assignee: YUR Brace, LLC, Dakota Dunes, SD (US)

(21) Appl. No.: 18/741,436

(22) Filed: Jun. 12, 2024

Related U.S. Application Data
(60) Provisional application No. 63/507,797, filed on Jun. 13, 2023.

(51) Int. Cl. A61F 5/02; A61F 5/058; A61M 35/00

(52) U.S. Cl. CPC A61F 5/028; A61F 5/05816; A61M 35/00; A61M 2205/05

(57) ABSTRACT

A portable brace assembly for therapeutically supporting an area of a user's body. The assembly generally includes a belt configured to be positioned around the user. One or more inflatable bladders are attached to the belt. One or more additional therapeutic or support modalities configured to be combined with the belt. The additional modalities may be positioned over the inflatable bladders, so that in use the additional modalities are positioned between the inflatable bladders and the user's body. A transdermal medication delivery assembly may be configured to be combined with the first side of the belt as an additional modality.

### BACKGROUND

This application is based upon U.S. Provisional Application Ser. No. 63/507,797 filed Jun. 13, 2023, the complete disclosure of which is hereby expressly incorporated by this reference.

This invention relates generally to belts and braces, and more particularly to a support brace for supporting a user's back, knee, elbow or other body part and providing therapeutic modalities thereto.

Low back pain commonly arises in individuals due to alterations in the biomechanics of the lumbosacral spine and its supporting muscular and ligamentous structures. Surgery may be required to address certain problems, and physical therapy may solve other problems. In addition, a physician can use a variety of orthotic devices such as braces to stabilize an injured or diseased body part before or after surgery. Orthotic devices are common to enhance and ensure healing following various types of surgery. The orthotic device may form an integral component of the treatment or recovery process. Therapeutic appliances can be useful, both in the treatment and prevention of back pain in individuals, when the primary mechanism involved in the generation of pain is due to muscular strain, spasm, or fatigue.

Known supports have been designed in an attempt to limit fatigue and undue strain in the above-mentioned structures and to aid in the restoration of both normal and balance muscle tone. While such aims are rarely achieved by the application of known therapeutic appliances alone, such appliances are often helpful in the prevention and treatment of various lower back conditions.

Known brace appliances are apt to exhibit one or more significant disadvantages. For example, existing braces require constrictive tightening of the device in order to provide support, hence causing uncomfortable and potentially detrimental compression of abdominal contents, especially for pregnant women. Existing braces require circumferential pressure and thus do not accommodate for scoliosis or other spinal plane anomalies. Another disadvantage and potentially harmful consequence of some braces is the required circumferential tightness to provide support to post-surgical patients. It is common practice to require and/or advise a patient to wear a brace after spine decompression and spine fusion surgery. Concentrated brace pressure thusly exerts significant pressure on the incision (near the midline of the spine) leading to irritation, pain, and even wound dehiscence leading to potential infection.

Another commonly applied modality of addressing musculoskeletal pain is the employment of a variety of topical/transdermal medicines such as gels, creams, ointment and medically infused pain-relieving patches. It is widely known that diffusion of pharmaceutical agents through the skin barrier requires at a minimum three minutes of massage (or by intense pressure) to permeate the degree structural elements of interest (muscles, tendons, ligaments, etc.). It can be difficult for patients to properly apply the medicine, especially when the medicine is applied to help pain in the patient's lower back. It can be difficult for patients to properly apply the medicine especially in elderly, single or compromised individuals.

There is therefore a need for an improved brace that overcomes these and other disadvantages in the prior art.

### SUMMARY

One aspect of the invention relates to a portable brace assembly for therapeutically supporting an area of a user's body wherein the user has a back, spine, side, waist, and belly. The assembly generally comprises a belt having a support portion between a first end portion and a second end portion. The first end portion is releasably connectable the second end portion for securing the belt around the user. The belt has a first side configured to face toward the user and a second side configured to face away from the user. The first side is radially inward from the second side when the belt is positioned around the user. The assembly further includes an inflatable bladder configured to be attached to the support portion of the belt and to expand from a non-inflated position to an inflated position upon the introduction of fluid. The inflatable bladder is positioned a first distance from the first end portion of the belt. In some embodiments the belt includes a longitudinal center and the inflatable bladder comprise a first bladder member on one side of the longitudinal center and a second bladder member on another side of the longitudinal center. In some embodiments the support portion and/or bladder is offset with respect to the longitudinal center. The assembly further includes a modality configured to be positioned radially inward from the inflatable bladder. The modality is positioned about the same (first) distance from the first end portion of the belt such that the modality is positioned between the inflatable bladder and the user when the belt is positioned around the user. The inflatable bladder is configured to urge the modality toward the user as the bladder is inflated from the non-inflated position to the inflated position.

Another aspect of the invention relates to a portable brace assembly for therapeutically supporting an area of a user's body. This assembly allows the deployment of multiple pain-relieving modalities that consider the unique biologic variability of individual patients. The assembly generally includes a belt configured to be positioned around the user, for example, around the waist of the user to support the user's lower back. The belt may be a band-like elastic material made from a soft fabric. The belt has a first side configured to face toward and engage the user's body and a second side configured to face away from the user's body. In some embodiments at least one of the first side of the belt and the second side of the belt include a fastener, such as hook and loop fabric (VELCRO), configured to removably attach to the components of the assembly described herein. In these embodiments either the hook material or the loop material is combined with the belt and the other of the hook material or the loop material is combined with the component of the assembly. One or more inflatable bladders are attached (directly or indirectly) to the first side of the belt. In some embodiments, the inflatable bladder is part of the belt or embodied within the belt. In some embodiments the inflatable bladders comprise two spaced-apart independently inflatable bladder members mounted (directly or indirectly) on the belt so that one bladder member is engageable with the lower back on one side of the spine and the second bladder member is engageable with the lower back on the opposite side of the spine. The inflatable bladders can be inflated with a manual pump or with an electric pump, which may communicate with a software application on a phone. In some embodiments the brace assembly further includes one or more additional therapeutic or support modalities configured to be combined (directly or indirectly) with the one or more inflatable bladders or the first side of the belt. The additional modalities may be positioned radially inward from the inflatable bladders, so that in use the additional modalities are positioned between the inflatable bladders and the user's body. In some embodiments a fastener, such as a layer of hook or loop fabric (VELCRO), is positioned over the bladders to provide a surface for attaching the additional modality. The one or more additional modalities may include medication dispensing assemblies, a TENS (transcutaneous electrical nerve stimulation) unit, copper pads, heating pads, vibration/messaging pads, cool gel packs, and magnets. A semi-rigid reinforcement member may be combined (directly or indirectly) with the belt. The semi-rigid reinforcement member provides a support surface for the bladders to push against while keeping the circumferential forces lower (more comfortable). The inflatable bladders, semi-rigid reinforcement member, and the additional modalities may be combined with the belt using a fastener, such as hook and loop fabric (VELCRO), or by any other suitable means so that the bladders and additional modalities may be removed from the belt or repositioned on the belt. At least one of the additional modalities may be used along with the inflatable bladders to provide the therapeutic effect of the additional modality while also providing the support of the inflatable bladders. In some embodiments the assembly further includes a sensor configured to communicate information to a processor (which may be the user's phone). The information may include the inflatable bladder pressures, the number or type of modalities attached to the belt, and the lengths of time the brace assembly and each modality was worn by the user.

Another aspect of the invention relates to a kit for a portable brace for therapeutically supporting an area of a user's body. The kit includes one or more of the features described above with the portable brace assembly, including a belt, one or more inflatable bladders, semi-rigid reinforcement member, a sensor configured to communicate information to a processor, and one or more additional modalities.

Another aspect of the invention relates to a transdermal medication delivery assembly configured to be combined (directly or indirectly) with the first side of the belt as an additional modality. The transdermal medication delivery assembly may be an absorbent pad, such as a foam pad, impregnated with medication. In some embodiments the transdermal medication delivery assembly is a pouch having a first side, a second side, and a medication reservoir. The first side has a semipermeable membrane configured to engage the user's skin. In some embodiments the semipermeable membrane is cloth fabric. The semipermeable member selectively releases medication onto the user's skin from the medication reservoir. The second side of the pouch includes a fastener for attaching the pouch to the belt. In some embodiments the fastener is hook and loop material (VELCRO) wherein either the hook material or the loop material is combined with the pouch and the other of the hook material or the loop material is combined with the belt so the transdermal medication pouch can be easily attached, removed, and repositioned on the belt. In some embodiments the transdermal medication pouch is aligned with one of the inflatable bladders so the inflatable the bladder is configured to apply pressure on the pouch when the belt is being worn by the user. In this manner the amount of medication applied to the patient's skin can be controlled by increasing or decreasing the volume in the bladders since increased pressure on the pouch from the bladders causes more medicine to be pushed through the semipermeable membrane onto the user's skin. Furthermore, different degrees of permeability of the membrane and various viscosities of the ointment will impact the rapidity of said medication delivery onto the body.

Another aspect of the invention relates to a method of using the assemblies described above. In use, the user attaches the inflatable bladders to a desired position on the first side of the belt. The user then attaches additional modalities (if any) to the first side of the belt. The belt is attached about the person's body with the bladder or bladders overlying the lumbar and sacral regions, and the bladder or bladders are inflated to provide the amount of pressure desired by the wearer. The pressure provides mechanical support for the muscles and ligaments in the lower spine area and provides static stretch to the erector spinae muscle and aids in the prevention and elimination of spasms by maintaining these muscles in a more stretched and relaxed state. The additional modality may be actuated and data may be collected by the sensor. The invention is described with respect to a back/spine brace, however, the various features and embodiments may be use with other types of braces, including knee, shoulder, wrist, and elbow.

### BRIEF DESCRIPTION OF THE DRAWINGS

FIG. 1 is a front view of an embodiment of the brace assembly.

FIG. 2 is a rear view of the brace assembly shown in FIG. 1.

FIG. 3 is a front view of another embodiment of the brace assembly.

FIG. 4 is a rear view of the brace assembly shown in FIG. 3.

FIG. 5 is a front view of another embodiment of the brace assembly.

FIG. 6 is a rear view of the brace assembly shown in FIG. 5.

FIG. 7A is an exploded view of the brace assembly shown in FIGS. 1 and 2 having the inflatable bladders and an additional modality combined therewith.

FIG. 7B is an exploded view of the brace assembly shown in FIGS. 5 and 6 having the inflatable bladders and an additional modality combined therewith.

FIG. 7C is a perspective view of an alternate embodiment of the semi-rigid reinforcement member.

FIG. 7D is a side view of the semi-rigid reinforcement member shown in FIG. 7C.

FIGS. 8A-8E show front views of exemplary additional modalities.

FIG. 9 is a front view of a transdermal medication assembly.

FIG. 10 is a section view of a transdermal medication assembly takin along line A-A of FIG. 9.

FIG. 11 is a functional block diagram illustrating the components of a general purpose computing system connected to a general purpose electronic network, such as a computer network that can be utilized with the sensors and processors described herein.

### DETAILED DESCRIPTION

The invention generally relates to a brace assembly having one or more support or treatment modalities 14. Although the invention is generally described with respect to a back brace, the various features and embodiments may be used with other types of braces, including braces for knees, shoulders, wrists, and elbows.

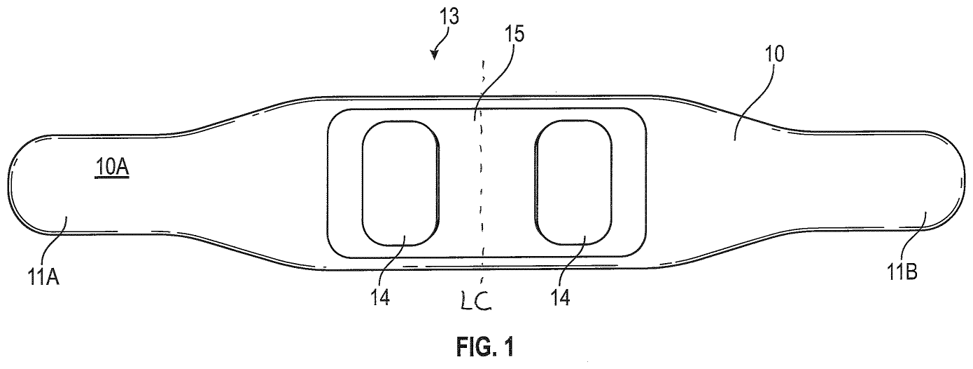

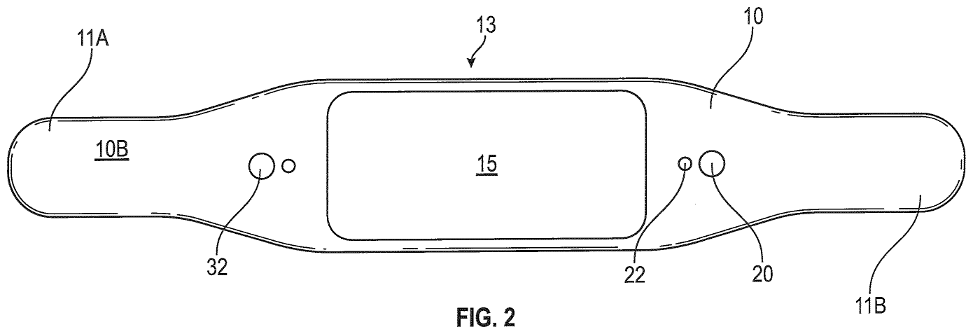

FIGS. 1 and 2 show a first embodiment of the invention wherein the brace assembly is a back brace. The assembly generally includes a belt 10 having two ends 11A, 11B on either side of a support portion 13. The assembly is configured to be positioned around the user, for example, around the waist of the user so the support portion 13 engages and supports the user's lower back. The belt 10 may be a band-like elastic material made from a soft fabric such as neoprene, polyesters, or spandex. The belt 10 and other components of the assembly may be made from or impregnated with an antimicrobial material, such as silver, zinc, or copper. In some embodiments the belt 10 is reinforced with a less flexible material such as fibers of polyester or nylon. The belt 10 has a first side 10A configured to face toward and engage the user's body and a second side 10B configured to face away from the user's body. In use, when the belt 10 is placed around the user, the first side 10A is radially inward from the second side 10B. In some embodiments at least one of the first side 10A of the belt 10 and the second side 10B of the belt 10 include a fastener, such as hook and loop fabric (VELCRO), configured to removably attach to the components on the assembly described herein. In embodiments using hook and loop material as the fastener, either the hook material or the loop material is combined with the belt 10 and the other of the hook material or the loop material is combined with one or more of the components described herein for removable attachment to the belt 10. The end portions 11A, 11B of the belt 10 are configured to be connected at a desired size in front of the user (near the user's hips/bellybutton) while the support portion 13 of the belt 10 is positioned near a lower portion of the user's back. In other embodiments the end portions 11A, 11B are configured to be connected at the user's side. In these embodiments the support portion 13 is offset with respect to a longitudinal center LC of the belt 10 so the support portion 13 is positioned near a lower portion of the user's back while the end portions 11A, 11B are connected at the user's side. One end 11A of the belt 10 can be secured at various locations along the other end 11B of the belt 10 whereby the tension of the elastic member can be varied to adjust the tension of the appliance and/or the force at which the assembly is urged into engagement with the lower back. In some embodiments the support portion 13 is wider than the end portions 11 to provide additional support at the desired area of the body/back.

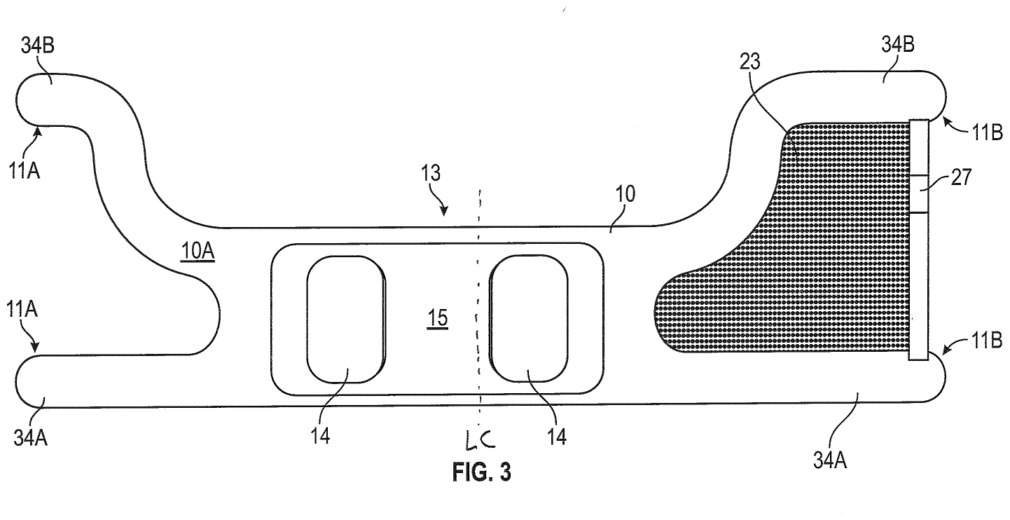

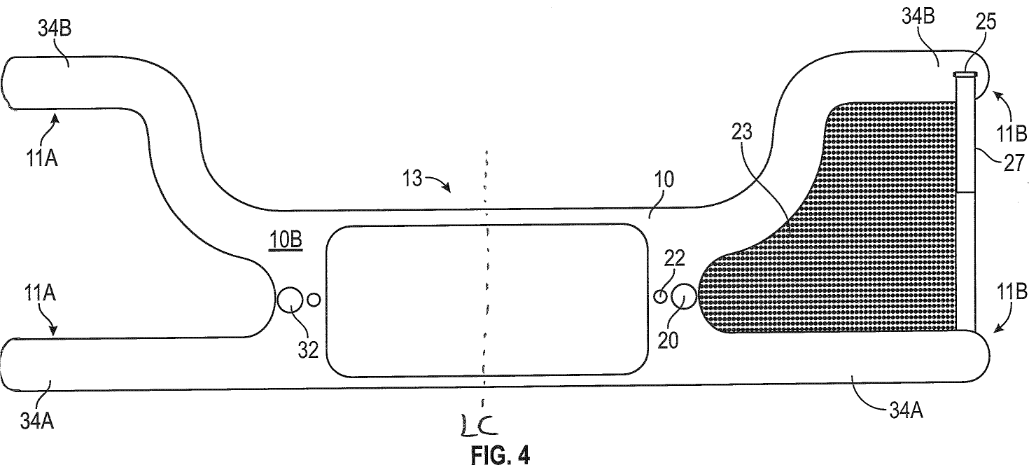

FIGS. 3 and 4 show an alternate embodiment of the belt 10 intended for use by people with large bellies, such as obese people and pregnant women. As shown, each end 11A, 11B of the belt 10 has a first (lower) portion 34A spaced a predetermined distance from a second (upper) portion 34B so there is an opening between the lower portion 34A and the upper portion 34B. The first portion 34A is configured to extend around the user's waist (similar to the position of the belt 10 in FIGS. 1 and 2) and the second portion 34B is configured to be positioned above the user's belly and closer to the user's chest. The first portions 34A of each end 11A, 11B are configured to be secured together around the user's body and the second portions 34B of each end 11A, 11B are configured to be secured together around the user's body. As shown in FIG. 3, the upper and lower portions 34A, 34B on one end 11A may be shorter than on the other end 11B so the support portion 13 is offset from the longitudinal center LC of the belt 10. This allows the end portions 11A, 11B of the brace assembly to be secured together at the user's side instead of at the user's front/belly while still placing the support portion 13 at the lower portion of the user's back.

The first (lower) portion 34A holds the brace pressure on the hip region while the second (upper) portion 34B is positioned above the apex of the belly. The compressive force at which the assembly is urged into engagement with the lower back is placed on the user's waist (below the belly) by the first portions 34A and above the user's belly by the second portions 34B. Force on the belly is minimized. The first portion 34A and the second portion 34B both connect to the support portion 13, and the two portions 34A, 34B on one side of the support portion 13 generally mirror the two end portions 34A, 34B on the other side of the support portion 13.

The embodiment shown in FIGS. 3 and 4 may further include a belly support element 23 between the belt first portion 34A and the belt second portion 34B. The belly support element 23 may be made from a material different from the belt 10. The belly support element 23 is configured to help support the user's belly by lifting the belly upward toward the belt second portion 34B. In some embodiments the belly support element 23 includes multiple layers. A first layer may be a soft cotton or spandex material positioned radially inward so it faces toward the user's body during use. A second layer is more rigid and is capable of providing more support for the belly. For example, the second layer may be a rope or fabric netting that does not stretch as much as the first layer. The lower portion of the belly support element 23 is attached to the belt first portion 34A by stitching, gluing, or other suitable means. The upper portion of the belly support element 23 is adjustably combined with the belt second portion 34B. A support member 27 such as a belt, rope, or cord, is attached to the first portion 34A and/or the belly support element 23 by stitching, gluing, or other suitable means. A portion of the support member 27 may be selectively fastened to the belt second portion 34B by a fastener 25 to help secure the belly support element 23 to the upper/second portion 34B of the belt. In this manner the belly support element 23 lifts the user's belly upward toward the send end portion 11B in a direction that is generally perpendicular to the radial support provided by the belt and the bladder members 12 (described below). In pregnant women, this helps to lift the baby off of the pubic bone to help eliminate a common source of pain for third trimester mothers (i.e. pubic pain). It also helps to positively alter the biomechanical load paced on the spine. Biomechanically, both in pregnant or obese individuals, this redirection of the center of gravity will restore proper axial load on the spine hence relieving a major sort of pain from stressed diseased facet joints. In some embodiments the fastener 25 may be an adjustable fastener to allow the user to change the amount of upward lift provided by the belly support element 23.

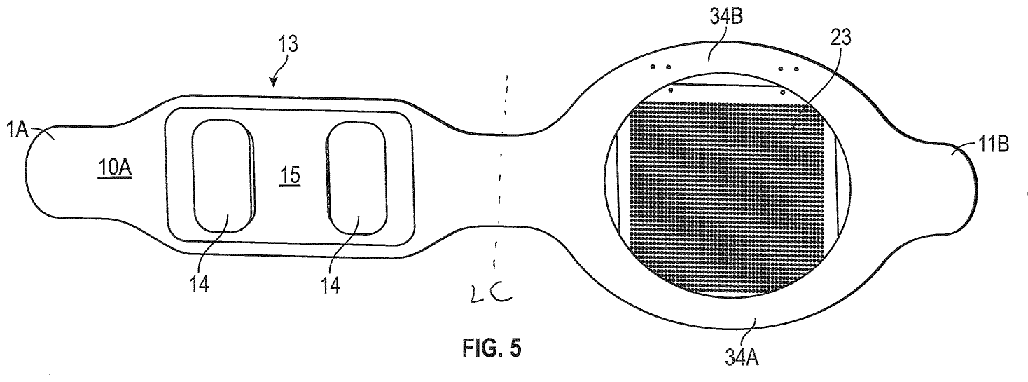

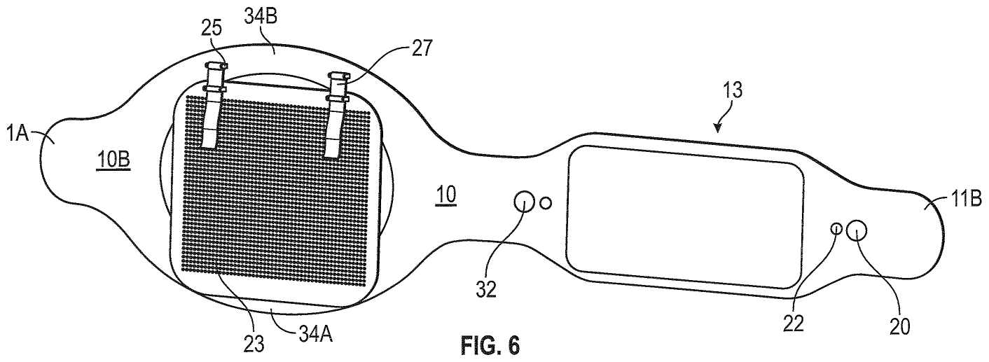

FIGS. 5 and 6 show an alternate embodiment similar to the embodiment shown in FIGS. 3 and 4. In the embodiment shown in FIGS. 5 and 6, the belt first portion 34A and the belt second portion 34B are connected together at both ends but spaced apart creating an opening therebetween. The opening is configured to receive the user's belly. In the embodiment shown, the belt first portion 34A and the belt second portion 34B form an oval shape with an oval opening therebetween. In other embodiments the belt first portion 34A and the belt second portion 34B form other suitable shapes. The support portion 13 is offset from the longitudinal center LC of the belt thereby allowing the end portions 11A, 11B of the brace assembly to be secured together at the user's side instead of at the user's front/belly while still placing the support portion 13 at the lower portion of the user's back. A belly support element 23 such as the one described above may also be used with the embodiment shown in FIGS. 5 and 6.

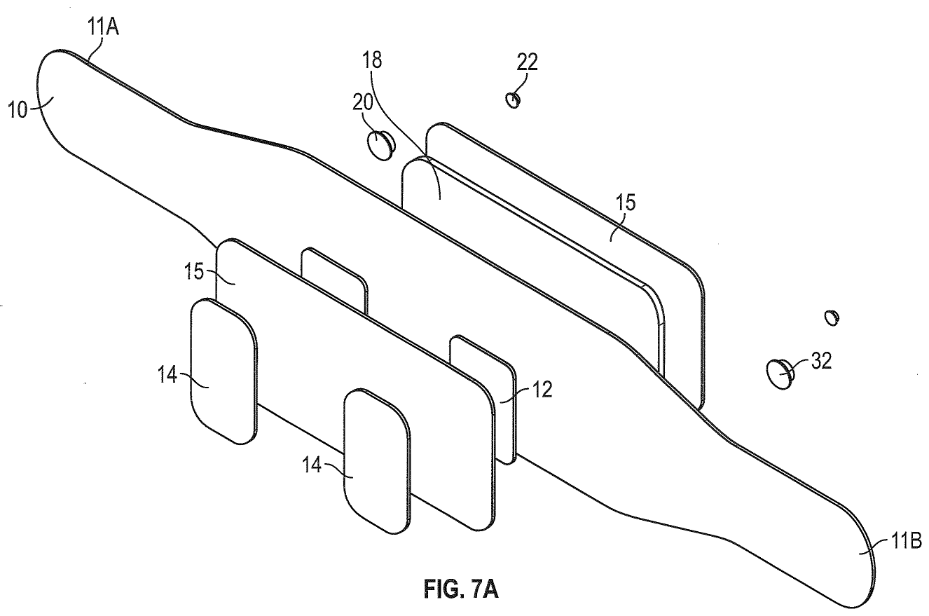

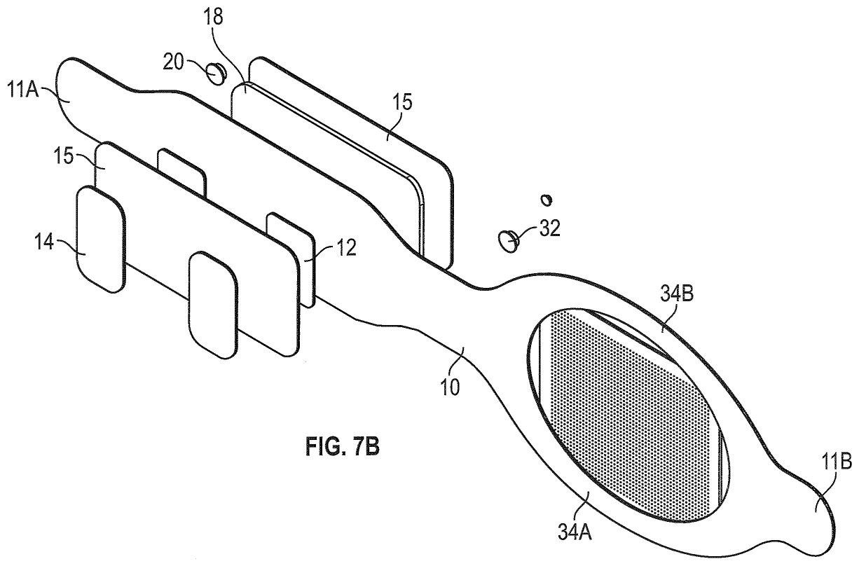

FIGS. 7A and 7B show exploded views of the brace assembly wherein the belt 10 is positioned between several other components. As shown, one or more inflatable bladders 12 are attached (directly or indirectly) to the support portion 13 of the belt 10 on the first side 10A of the belt 10. In some embodiments the bladders 12 are part of or embedded within the belt 10. In some embodiments the inflatable bladders 12 comprise two spaced-apart independently inflatable bladder members 12 mounted (directly or indirectly) on the belt 10 so that one bladder member 12 is engageable with the lower back on one side of the spine and the second bladder member 12 is engageable with the lower back on the opposite side of the spine. The inflatable bladders 12 can be inflated by any suitable means and they may be inflated independently to different sizes/pressures. Inflating the bladders 12 to different sizes/pressures is useful in many situations, including where patients have scoliosis. The effect of this degenerative condition often leads to half of the lower back being concave while the other half is convex. As shown, a pump 20 is combined with the assembly to inflate the bladders 12. A tube or other conduit (not shown) may connect the pump 20 to the bladders 12. The pump 20 may be a manual pump or it may be electric pump. Some embodiments may include a switch 22 configured to inflate or deflate fluid to/from the inflatable bladders 12. In some embodiments the switch 22 is in communication with a valve so that pressing the switch 22 releases fluid from the bladders 12. Each bladder 12 may have its own pump 20 and switch 22 so fluid in each bladder 12 can be independently adjusted and maintained. In embodiments wherein the pump 20 is electric, the pump 20 may be in communication with a software application on a phone or other device to control the pressure (inflation/deflation) in each bladder 12.

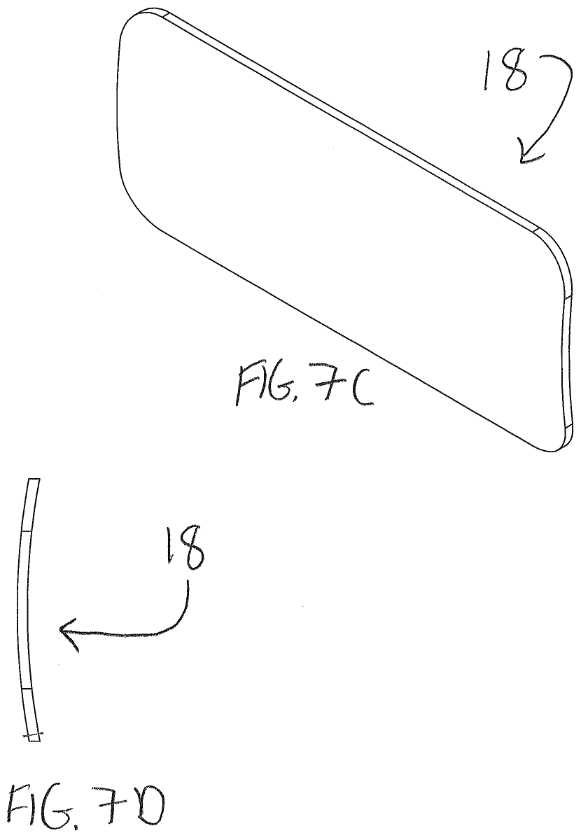

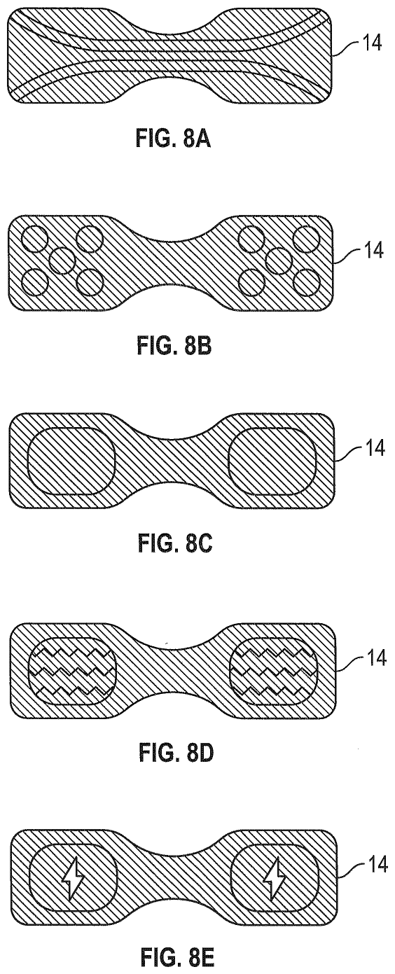

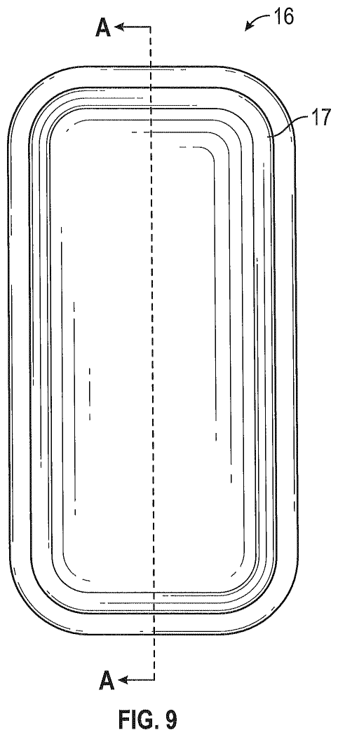

As shown in FIGS. 7A and 7B, the brace assembly further includes one or more additional therapeutic or support modalities 14 configured to be combined (directly or indirectly) with the first side 10A of the belt 10. The additional modalities 14 may be positioned over (radially inward from) the inflatable bladders 12, so that in use the additional modalities 14 are positioned between the inflatable bladders 12 and the user's body. In some embodiments a fastener 15, such as a layer of hook and loop fabric (VELCRO), is positioned between the bladders 12 and the additional modality 14 (over the bladders 12). The fastener 15 provides a surface for attaching the additional modality 14, which may have a corresponding fastener (hook or loop material) combined thereto. As shown in FIGS. 8A-8E, the one or more additional modalities 14 may include copper infused fabric (FIG. 8A), magnet infused fabric (FIG. 8B), transdermal medication delivery assembly such as an absorbent pad, such as a foam pad, impregnated with medication, or a delivery pouch (FIG. 8C), vibration/messaging pads (FIG. 8D), a TENS unit (FIG. 8E), a pouch or “fanny pack”, or other suitable modalities, such as heating pads. The heating pads may be chemical heat packs generating heat as a result of a chemical reaction, reusable heat packs which generate heat as a result of being heated (such as by heating the pack in the microwave), or electric heat packs which generate heat by way of electric/battery power. At least one of the additional modalities 14 may be used along with the inflatable bladders 12 to provide the therapeutic effect of the additional modality 14 while also providing the support of the inflatable bladders 12. Further, using the inflatable bladders 12 along with the additional modality 14 allows the user to control the firmness or force at which the additional modality 14 is pressed against the user's body. Increased inflation of the bladders 12 results in increased pressure of the modality 14 against the user's body. Conversely, reduced inflation of the bladders 12 results in less pressure of the modality 14 against the user's body.

In some embodiments the components of the assembly are water proof or water resistant thereby allowing use of the device in the water or in wet environments, such as when sailing, kayaking, paddle boarding, operating a personal water craft, or hunting in wet conditions. In one embodiment the individual electrical components and connections are water proof or water resistant. In another embodiment some or all of the assembly is enclosed within a water proof or water resistant sleeve.

A semi-rigid reinforcement member 18 may be combined (directly or indirectly) with the second side 10B of the belt 10 using any suitable fastener, including hook and loop material. FIG. 7A shows the semi-rigid reinforcement member 18 as a generally flat panel positioned radially outward from the bladders 12. The semi-rigid reinforcement member 18 provides a relatively firm surface against which the bladders 12 may press as they inflate. This allows for more support while keeping the radial/circumferential forces lower (more comfort). In some embodiments, the belt 10 includes pockets configured to selectively receive one or more semi-rigid reinforcement members 18. In other embodiments, the semi-rigid reinforcement members 18 are combined with the belt by VELCRO or other suitable means. FIGS. 7C and 7D show an alternate embodiment wherein the semi-rigid reinforcement member 18 has a curved shape to help increase stiffness while minimizing thickness and weight. Further, the curve may increase comfort by reducing the user's ability to feel the edges of the reduce the semi-rigid reinforcement member 18 since the convex side with the outwardly extending curve faces toward the user's back and the top and bottom edges curve away from the user's back.

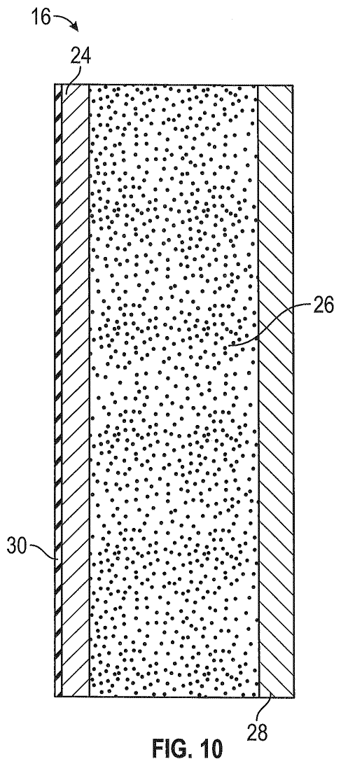

As noted above, one of the additional modalities 14 may be a transdermal medication delivery assembly 16 configured to be combined (directly or indirectly) with the first side 10A of the belt 10. FIGS. 9 and 10 show an embodiment of the transdermal medication delivery assembly 16. The transdermal medication delivery assembly 16 has a first side, a second side, and a medication reservoir 26. The first side has a semipermeable membrane 24 configured to engage the user's skin after the protective seal 30 is peeled off. In some embodiments the semipermeable membrane 24 is a fabric or cloth material. The semipermeable membrane 24 selectively releases medication onto the user's skin from the medication reservoir 26. The medication is preferably a liquid, gel, oil, or cream capable passing through the membrane 24 by osmosis facilitated by the pressure of the bladders 12. The medication may include lidocaine, diclofenac, capsaicin, salicylic acid, eucalyptus oil, camphor, and CBD extracts. The second side of the pouch 16 includes a fastener 28 for attaching the pouch 16 to the belt 10. As explained above, for this and other additional modalities 14 the fastener 28 may be hook and loop material (VELCRO) so the transdermal medication pouch 16 can be easily attached, removed, and repositioned on the belt 10 or on some intermediate structure, such as the attachment layer 15 described above.

As shown in FIG. 9, some embodiments of the transdermal medication delivery assembly 16 include a flexible gasket 17 on the first side of the assembly 16. The gasket 17 is positioned around the periphery of the semi-permeable membrane 24 and is configured to engage the user's skin to provide a barrier which helps keep the medication contained as the medication moves through the semi-permeable membrane 24. The gasket 17 helps prevent the medication from leaking or migrating away from the desired application area. In some embodiments the gasket 17 is made from a rubber or silicone material.

In another embodiment the transdermal medication delivery assembly 16 may be an absorbent pad, such as a foam pad, impregnated with medication. The absorbent pad may be similar to the semi-permeable membrane 24 described above. This embodiment is similar to the embodiment described above with respect to FIGS. 9 and 10, but does not include any separate medication reservoir 26.

As shown in FIGS. 7A, 7B, the transdermal medication delivery assembly 16 may be one of the additional modalities 14 attached to the belt 10 radially inward from the bladders 12. The medication delivery assembly 16 may be positioned over one of the inflatable bladders 12 so the inflatable the bladder 12 is configured to apply pressure on the delivery assembly 16 when the belt 10 is being worn by the user. In this manner the amount of medication applied to the patient's skin can be controlled by increasing or decreasing the volume in the bladders 12 since increased pressure on the pouch 16 from the bladders 12 causes more medicine to be pushed through the semipermeable membrane 24 onto the user's skin. By employing sufficient pressure via the air bladder system, medication will be pushed through the permeable membrane 24 of the medication pouch, and trans-dermal infusion into the designated anatomical location on the patient.

In some embodiments, more than one additional modality 14 may be used at the same time. Each modality 14 may have hook and loop attachment means on one or both sides so the modalities 14 can be stalked on top of each other. As discussed above, it is desirable to align the modalities 14 with the bladder 12 so adjustment of the pressure in the bladders 12 helps control the desired amount of pressure the modalities 14 apply against the user's body. In one embodiment, for example, a heat modality 14 and a messaging modality 14 may be used along with a medication pouch 16. In this exemplary embodiment the bladders 12 are farthest from the user's body, then the messaging modality 14, heat modality 14, and finally the medication pouch 16 adjacent to the user's skin.

In some embodiments the assembly further includes electronics configured to control components of the assembly or communicate with other electronic components (such as phones, tablets, or computers). As shown in FIGS. 2, 4, 6, 7A, and 7B, the assembly may include a sensor 32 configured to detect or measure certain properties of the assembly and communicate the information to a processor (which may be the user's phone). In some embodiments the sensor 32 is configured to detect or measure the inflation pressure of each inflatable bladder 12, which additional modality 14 is attached to the belt 10, and the lengths of time the brace assembly and additional modality 14 was worn by the user. The sensor 32 may communicate the information to a processor that is separate from the brace assembly. For example, the sensor 32 may communicate via Bluetooth or RFID to the user's phone. The processor/phone may run a software application configured to control the inflatable bladders 12 or other additional modalities 14. The processor/phone may store certain preset conditions (i.e., bladder pressure) and control the bladder members 12 and additional modalities 14 accordingly. In some embodiments the processor/phone monitors the pressure in the bladder member 12 to ensure they are not overinflated. The processor/phone may also be configured to communicate data to a central database where patient data is gathered to improve future versions of the brace assembly.

In use, the user attaches the inflatable bladders 12 to a desired position on the first side of the belt 10. The user then attaches additional modalities 14 (if any) to the first side of the belt 10. The belt 10 is attached about the person's body with the bladder or bladders 12 overlying the lumbar and sacral regions, and the bladder or bladders 12 are inflated to provide the amount of pressure desired by the wearer. The pressure provides mechanical support for the muscles and ligaments in the lower spine area and provides static stretch to the erector spinae muscle and aids in the prevention and elimination of spasms by maintaining these muscles in a more stretched and relaxed state. The additional modality 14 may be actuated and data may be collected by the sensor. The invention is described with respect to a back brace, however, the various features and embodiments may be use with other types of braces, including knee, shoulder, wrist, and elbow.

Patients can opt in to collecting data and sending it for research. User can receive advertising, reorder medical delivery assemblies 16 and other additional modalities 14 over the software application.

It will be appreciated that some embodiments of the present invention may be implemented with a computer or computer-based network. A computer 14 may be specifically programmed to carry out the steps described above and store information related thereto. For example, a computer may be used to store data related to the brace assembly and any additional modalities. Thus, embodiments within the scope of the present invention include program products comprising computer-readable media for carrying or having computer executable instructions or data structures stored thereon. Such computer-readable media can be any available media that can be accessed by a general purpose or special purpose computer. By way of example, such computer-readable media can comprise RAM, ROM, EPROM, EEPROM, CD-ROM or other optical storage, magnetic disk storage or other magnetic storage devices, or any other medium which can be used to carry or store desired program code in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above are also to be included within the scope of computer-readable media. Computer-executable instructions comprise, for example, instructions and data which cause a general purpose computer, special purpose computer, or special purpose processing device to perform a certain function or group of functions.

The present invention in some embodiments, may be operated in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. In some embodiments, users may be able to access the network to provide and receive information about the brace assembly and its additional modalities 14.

The invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of the invention might include a general-purpose computing device in the form of a conventional computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to removable optical disk such as a CD-ROM or other optical media. The drives and their associated computer-readable media provide nonvolatile storage of computer-executable instructions, data structures, program modules and other data for the computer.

Software and Web implementations of the present invention could be accomplished with standard programming techniques with rule based logic and other logic to accomplish the various database searching steps, correlation steps, comparison steps and decision steps. It should also be noted that the words “component” or “module” as used herein is intended to encompass implementations using one or more lines of software code, and/or hardware implementations, and/or equipment for receiving manual inputs.

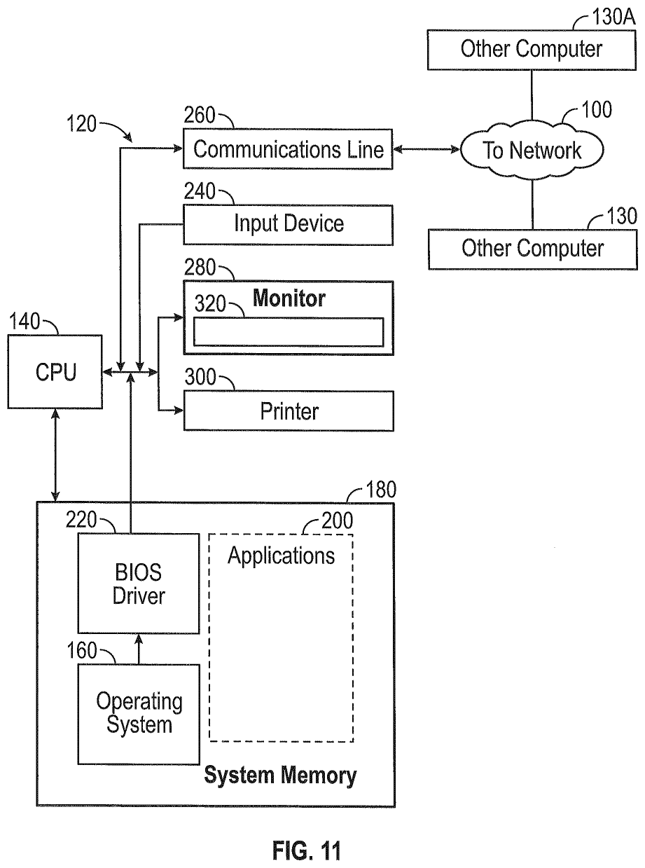

FIG. 11 illustrates the components of a general-purpose computing system connected to a general-purpose electronic network 100, such as a computer network. The computer network can be a virtual private network or a public network, such as the Internet. As shown in FIG. 200, the computer system 120 includes a central processing unit (CPU) 140 connected to a system memory 180. The system memory 180 typically contains an operating system 160, a BIOS driver 220, and application programs 200. In addition, the computer system 120 contains input devices 240 such as a mouse or a keyboard 32, and output devices such as a printer 300 and a display monitor 280, and a permanent data store, such as a database 210. The computer system generally includes a communications interface 260 to communicate to the electronic network 100. Other computer systems 130 and 130A also connect to the electronic network 100 which can be implemented as a Wide Area Network (WAN) or as an internetwork, such as the Internet. Data is stored either in many local repositories and synchronized with a central warehouse optimized for queries and for reporting, or is stored centrally in a dual use database. This system is one example of a system that could execute the method steps set forth above.

Having thus described the invention in connection with the preferred embodiments thereof, it will be evident to those skilled in the art that various revisions can be made to the preferred embodiments described herein without departing from the spirit and scope of the invention. It is my intention, however, that all such revisions and modifications that are evident to those skilled in the art will be included with in the scope of the following claims.

What is claimed is as follows:

1. A portable brace assembly configured to be positioned around a user, the user having a back, spine, side, waist, and belly, said assembly comprising:
a belt having a support portion between a first end portion and a second end portion, a first side configured to face toward the user and a second side configured to face away from the user, wherein the first end portion is releasably connectable the second end portion and the first side is radially inward from the second side when the belt is positioned around the user;
an inflatable bladder configured to be attached to the support portion of the belt and to expand from a non-inflated position to an inflated position upon the introduction of fluid, the inflatable bladder positioned a first distance from the first end portion of the belt; and
a modality configured to be positioned radially inward from the inflatable bladder, the modality positioned about the first distance from the first end portion of the belt such that the modality is positioned between the inflatable bladder and the user when the belt is positioned around the user, wherein the inflatable bladder is configured to urge the modality toward the user as the bladder is inflated from the non-inflated position to the inflated position.

2. The portable brace assembly of claim 1 wherein the modality includes one or more of: a medication dispensing assembly, a TENS (transcutaneous electrical nerve stimulation) unit, a copper pad, a heating pad, a vibration/messaging pad, a cool gel pack, and a magnet.

3. The portable brace assembly of claim 1 further comprising a fastener positioned radially inward from the inflatable bladder for attaching the modality over the inflatable bladder.

4. The portable brace assembly of claim 3 wherein the fastener is hook and loop fabric.

5. The portable brace assembly of claim 1 further comprising a pump in fluid communication with the inflatable bladder and configured to introduce fluid into the inflatable bladder to inflate the inflatable bladder.

6. The portable brace assembly of claim 5 wherein the pump is configured to communicate with a software application so the pump can be controlled from the software application.

7. The portable brace assembly of claim 5 wherein the pump is an electric pump.

8. The portable brace assembly of claim 1 wherein the belt includes a longitudinal center and the inflatable bladder comprise a first bladder member on one side of the longitudinal center and a second bladder member on another side of the longitudinal center.

9. The portable brace assembly of claim 1 wherein the inflatable bladder comprise a first bladder member and a second bladder member, the first bladder member configured to engage the back on one side of the spine and the second bladder member configured to engage the back on another side of the spine.

10. The portable brace assembly of claim 1 wherein the belt includes a longitudinal center, wherein the support portion is offset with respect to the longitudinal center.

11. The portable brace assembly of claim 1 wherein the support portion is closer to the first end portion than the second end portion.

12. The portable brace assembly of claim 1 further comprising a semi-rigid reinforcement member combined with the belt radially outward from the inflatable bladder, said semi-rigid reinforcement member configured to provide a support surface for the inflatable bladder to push against.

13. The portable brace assembly of claim 12 wherein the semi-rigid reinforcement member is curved.

14. The portable brace assembly of claim 1 further comprising a sensor configured to measure data and communicate the data to a processor, wherein the data includes one of bladder pressure, modality information, and information about the length of time the brace assembly is worn by the user for a given period.

15. The portable brace assembly of claim 1 wherein the inflatable bladder is indirectly combined with the first side of the belt and the modality is indirectly combined with the inflatable bladder.

16. The portable brace assembly of claim 1 wherein the belt has a lower portion spaced a predetermined distance below an upper portion, the lower portion is configured to extend around the waist and the upper portion is configured to be positioned above the belly.

17. The portable brace assembly of claim 16 further comprising a belly support element between the lower portion and the upper portion, wherein the belly support element is configured to help support the belly by lifting the belly upward toward the upper portion.

18. The portable brace assembly of claim 17 further comprising a support member attached to the upper portion and the belly support element to help lift and secure the belly support element to the upper portion.

19. The portable brace assembly of claim 17 wherein the belt has a longitudinal center, the inflatable bladder is on one side of the longitudinal center and the belly support element is on another side of the longitudinal center.

20. The portable brace assembly of claim 1 wherein the modality is a transdermal medication delivery assembly having a first side, a second side, and a medication reservoir, the first side having a semipermeable membrane, the second side having a fastener configured to selectively attach the transdermal medication delivery assembly to the portable brace assembly.

21. A portable brace assembly configured to be positioned around a user, the user having a back, spine, side, waist, and belly, said assembly comprising:
a belt having a first end portion releasably connectable a second end portion, a first side configured to face toward the user and a second side configured to face away from the user, the first side being radially inward from the second side when the belt is positioned around the user, wherein the belt has a lower portion spaced a predetermined distance below an upper portion, the lower portion is configured to extend around the waist and the upper portion is configured to be positioned above the belly;
a belly support element between the lower portion and the upper portion, wherein the belly support element is configured to help support the belly by lifting the belly upward toward the upper portion;
an inflatable bladder configured to be attached to the first side of the belt and to expand from a non-inflated position to an inflated position upon the introduction of fluid.

22. The portable brace assembly of claim 21 wherein the belt has a longitudinal center, and the inflatable bladder is on one side of the longitudinal center and the belly support element is on another side of the longitudinal center.

23. The portable brace assembly of claim 21 wherein the inflatable bladder is indirectly combined with the first side of the belt.

24. The portable brace assembly of claim 21 wherein the belt is made from a stiffer material than the belly support element.

25. A portable brace assembly configured to be positioned around a user, said assembly comprising:
a belt having a first end portion releasably connectable a second end portion, a first side configured to face toward the user and a second side configured to face away from the user, the first side being radially inward from the second side when the belt is positioned around the user;
an inflatable bladder configured to be attached to the first side of the belt and to expand from a non-inflated position to an inflated position upon the introduction of fluid; and
a modality configured to be positioned radially inward from the inflatable bladder, wherein the modality is a transdermal medication delivery assembly having a first side, a second side, and a medication reservoir, the first side having a semipermeable membrane, the second side having a fastener configured to selectively attach the transdermal medication delivery assembly to the inflatable bladder.

26. The portable brace assembly of claim 25 wherein the semipermeable membrane is cloth fabric.

27. The portable brace assembly of claim 25 wherein the inflatable bladder is indirectly combined with the first side of the belt and the modality is indirectly combined with the inflatable bladder.

28. The portable brace assembly of claim 25 further comprising a gasket around a periphery of the semi-permeable membrane, the gasket configured to engage the user to provide a barrier which helps keep the medication contained as the medication moves through the semi-permeable membrane.

29. A kit for a portable brace assembly configured to be positioned around a user, the user having a back, spine, side, waist, and belly, said kit comprising:
a belt having a first end portion releasably connectable a second end portion, a first side configured to face toward the user and a second side configured to face away from the user, the first side being radially inward from the second side when the belt is positioned around the user;
an inflatable bladder configured to be attached to the first side of the belt and to expand from a non-inflated position to an inflated position upon the introduction of fluid;
a pump in fluid communication with the inflatable bladder and configured to introduce fluid into the inflatable bladder to inflate the inflatable bladder; and
a modality configured to be positioned radially inward from the inflatable bladder, wherein the modality is a transdermal medication delivery assembly having a first side, a second side, and a medication reservoir, the first side having a semipermeable membrane, the second side having a fastener configured to selectively attach the transdermal medication delivery assembly to the portable brace assembly.

30. The kit of claim 29 wherein the inflatable bladder is indirectly combined with the first side of the belt and the modality is indirectly combined with the inflatable bladder.

31. The kit of claim 29 further comprising a fastener positioned radially inward from the inflatable bladder for attaching the modality.

32. The kit of claim 27 further comprising a semi-rigid reinforcement member combined with the belt radially outward from the inflatable bladder, said semi-rigid reinforcement member is curved and configured to provide a support surface for the inflatable bladder to push against.

33. The kit of claim 27 wherein the belt has a lower portion spaced a predetermined distance below an upper portion, the lower portion is configured to extend around the waist and the upper portion is configured to be positioned above the belly, and the kit further comprises a belly support element to help lift and secure the belly support element to the upper portion.
